(19) United States
(12) Patent Application Publication
(10) Pub. No.: US 2026/0033715 A1
(43) Pub. Date: Feb. 5, 2026

(54) Gap Spectrum OCT

(71) Applicant: Carl Zeiss Meditec, Inc., Dublin, CA (US)

(72) Inventor: Matthew J. Everett, Livermore, CA (US)

(73) Assignee: Carl Zeiss Meditec, Inc., Dublin, CA (US)

(21) Appl. No.: 19/288,320

(22) Filed: Aug. 1, 2025

Related U.S. Application Data
(60) Provisional application No. 63/679,548, filed on Aug. 5, 2024.

(51) Int. Cl. A61B 3/10; A61B 3/12

(52) U.S. Cl. CPC A61B 3/102; A61B 3/1225

(57) ABSTRACT

A system, method and/or device for obtaining interferometric measurements using multiple light sources to image, in sequence, a location on a sample, where the light sources have different spectral wavelength bands, and adjacent spectral wavelength bands overlap each other. The respective interferometric measurements are combined mathematically to define a composite interferometric measurement for the location on the sample. The composite interferometric measurement has a greater axial resolution than that of any of the individual light sources.

### CROSS-REFERENCE TO RELATED APPLICATIONS

This application claims priority to, and the benefit of, U.S. Provisional Patent Application No. 63/679,548, filed Aug. 5, 2024 and titled “GAP SPECTRUM OCT,” which is incorporated by reference herein in its entirety for all purposes.

### FIELD

The present disclosure is generally directed to interferometric measurement systems. More specifically, the disclosure is directed to a swept source OCT system utilizing multiple light sources, each of which may be comprised of multiple sub light sources.

### BACKGROUND

Optical coherence tomography (OCT) is a cross-sectional, non-invasive imaging modality, which has application in diverse areas of medical imaging. In ophthalmology, OCT has been widely used for imaging the retina, choroid and anterior segment. Functional imaging of the blood velocity and vessel microvasculature is also possible. Fourier-domain OCT (FD-OCT) has high sensitivity and imaging speed compared to time-domain OCT (TD-OCT) embodiment, which uses an optical delay line for mechanical depth scanning with a relatively slow imaging speed. The spectral information discrimination in FD-OCT is typically accomplished either by using a dispersive spectrometer in the detection arm (spectral domain or SD-OCT) or rapidly scanning a swept laser source (swept-source OCT or SS-OCT).

Compared to spectrometer-based FD-OCT, swept-source OCT (SS-OCT) has many advantages, including its robustness to motion artifacts and fringe washout, lower sensitivity roll-off and higher detection efficiency, etc. Many different approaches have been implemented to develop high-speed swept sources, including semiconductor optical amplifier (SOA) based ring laser designs and short cavity lasers among others. Swept source OCT is highly desired by doctors, but the cost of an SS-OCT's broad band light sources limits the market to high end devices. Indeed, high resolution SS-OCT systems have heretofore been expensive. Various approaches at addressing limitations of OCT systems have previously been made. For example, U.S. Pat. Nos. 7,126,693 and 10,571,243 (herein incorporated in their entirety by reference and both assigned to the same assignee as the present disclosure), respectively provide configurations for improved efficiency OCT reflector systems and sequential angle illumination for ultra-high resolution OCT images. Insight Photonics (or Insight Photonic Solutions, Inc.™) provide an approach that physically combines multiple narrower bandwidth lasers, using laser control to make sure there are no overlapping spectra or gaps in the spectra. Additional examples are found in U.S. Pat. Nos. 8,724,667, 8,873,066, and 9,455,549, all of which are herein incorporated in their entirety by reference.

The present disclosure provides a low cost swept source OCT system. The present disclosure also provides a solution that overcomes the need for expensive, broad band light sources.

### SUMMARY

This method, device and/or system makes it possible to replace the expensive, broad band light source (e.g., such as used in a typical swept-source OCT) with multiple inexpensive narrower-band light sources, enabling cost-effective swept source instruments.

Today's OCT systems typically operate with an axial resolution of roughly 5 μm, using light provided by a broad bandwidth source. For swept source OCT, the light source is tunable and sweeps sequentially through all of the wavelengths within a bandwidth of interest. Achieving a swept source with the broad bandwidth used for 5 μm resolution is expensive, but narrower bandwidth lasers are significantly less expensive. However, combining multiple narrow bandwidth lasers to achieve the desired spectrum (e.g., the broad bandwidth desired for 5 μm resolution) is difficult. Some difficulties associated with combining multiple narrow bandwidth lasers to define the desired broad bandwidth to achieve 5 μm (or deeper in a axial direction) resolution are: any gaps in the spectra of the individual (narrow bandwidth) lasers lead to undesirable sidelobes in the OCT axial resolution function; any overlap in the spectra create challenges both in the combining and separating of the light from the (narrow bandwidth laser) light sources; and issues with the detection if the overlapping wavelengths are incident upon the detector simultaneously. Here, we have identified a way to combine the signals from multiple lasers with overlapping spectra without physically combining the beams, making it possible to create OCT images from multiple narrow band swept sources with overlapping spectra.

In essence, the present system/method/device obtains interferometric measurements using multiple light sources to image, in sequence, a location on a sample, where the light sources have different spectral wavelength bands, and adjacent spectral wavelength bands overlap each other. The respective interferometric measurements are combined mathematically to define a composite interferometric measurement for the location on the sample. This composite interferometric measurement has a greater axial resolution than that of any of the individual light sources alone.

The above features are included in a coherent interferometric measurement system, having: two or more light sources each emitting a different spectral wavelength band, (and may include a first light source and a second light source). The coherent interferometric measurement system includes one or more beam divider for directing a first portion of each of the two or more light sources into one or more reference arm and a second portion of the two or more light sources into a sample arm; optics for directing the light in the sample arm onto a sample; one or more detector for receiving light returning from the sample and reference arms and generating signals in response thereto; and a processor for converting the signals into image data. In various embodiments, there is overlap between the spectral wavelength bands of the two or more light sources; a location on a sample is illuminated with a first of the two or more light sources (e.g., the first light source), followed by illumination with another of the two or more light sources (e.g., the second light source); and the light returning from the sample from (due to) each of the two or more light sources is measured with a coherent interferometric measurement system (such as an OCT).

In various embodiments, the two or more measurements are summed coherently (e.g., including at least one of amplitudes of spectrums, complex sum of spectrum or OCT amplitude if converted to complex data). This combines the two or more measurements, and the combined measurement provides a higher axial resolution than either measurement alone.

In various embodiments, the light sources sweep their wavelength across their respective spectral wavelength bands. The wavelength sweeping is non-linear in k, and the collected data is remapped to create spectra that are linear in k. The linearization in k may be done prior to adding the signals together.

Additionally, the spectral overlap regions may be used to measure a phase offset between the two signals. This phase offset is corrected prior to the summing of the two signals.

The at least one of the two or more light sources consists of at least two sub-sources each having a different respective spectrum. In various embodiments, there is no overlap in the spectral bandwidth between the at least two sub-sources. Again, the two measurements are combined to provide a higher axial resolution than either measurement alone. In various embodiments, there is no overlap in the spectral bandwidth between the at least two sub-sources, but there is an overlap between the spectral bandwidth of at least one of the at least two sub-sources and another of the two or more light sources.

In the above approach, the optics for directing the light in the sample includes a scanner; the second portions of the two or more light sources are brought onto the scanner with an angular displacement between them, each second portion defining a respective sample beam, spatially offset from each other, along the sample arm. In various embodiments, the respective sample beams contact the sample at different corresponding offset locations; and as the respective sample beams are scanned, each sample beam follows the path of another sample beam so that the sample beams traverse the same locations on the sample so that each offset location is scanned sequentially by a plurality of the sample beams.

A light source may consist of one or more VCSELs. For example, the wavelengths of the VCSELs may be swept through (by) thermal heating, or through (by) movement of a membrane mirror. A light source may also consist of one or more distributed-feedback laser (DFB) or Distributed Bragg reflector (DBR) tunable laser.

In various embodiments, each of the one or more detectors receives respective light returning from the sample and reference arms corresponding to a respective one of the two or more light sources.

The above features are also included in a coherent interferometric measurement system, having: a first light source for generating a first beam of light having a first spectral wavelength band; a second light source for generating a second beam of light having a second spectral wavelength band different than the first spectral wavelength band, and the second spectral wavelength band overlapping the first spectral wavelength band; optics for directing at least a portion of the first beam to illuminate a target location on a sample, and for directing at least a portion of the second beam to illuminate the target location on the sample following illumination of the target location by the first beam in sequence; one or more detectors for receiving light returning from the target location on the sample due to the first beam and generating first signals in response thereto, and for receiving light returning from the target location on the sample due to the second beam and generating second signals in response thereto; and a processor for mathematically combining the first signals and second signals into a combined interferometric measurement for the target location on the sample, and converting the combined interferometric measurement into image data.

The present coherent interferometric measurement system may be an optical coherence tomography (OCT) system. The OCT system may also include one or more beam dividers (e.g., beam splitters or fiber coupler) for directing a first portion of the first beam into a first reference arm and a second portion of the first beam into a first sample arm, and for directing a first portion of the second beam into a second reference arm and a second portion of the first beam into a second sample arm; the optics direct second portion of the first beam in the first sample arm to illuminate the target location, and direct second portion of the second beam in the second sample arm to illuminate the target location; and the one or more detectors generate the first signals in response to returning light in the first sample arm and first reference arm, and generate second signals in response to returning light in the second sample arm and second reference arm. The first reference arm may be different than the second reference arm, such as that the OCT system has multiple reference arms. Also, the first sample arm and the second sample arm may share optical components and light path to the target location on the sample. For example, the first and second sample arms may be the same sample arm and the beam divider directs light to the same sample arm via one or more sample path.

Other features and attainments together with a fuller understanding of the disclosure will become apparent and appreciated by referring to the following description and claims taken in conjunction with the accompanying drawings.

Several publications may be cited or referred to herein to facilitate the understanding of the present disclosure. All publications cited or referred to herein, are hereby incorporated herein in their entirety by reference.

The embodiments disclosed herein are only examples, and the scope of this disclosure is not limited to them. Any embodiment feature mentioned in one claim category, e.g. system, can be claimed in another claim category, e.g. method, as well. The dependencies or references back in the attached claims are chosen for formal reasons only. However, any subject matter resulting from a deliberate reference back to any previous claims can be claimed as well, so that any combination of claims and the features thereof are disclosed and can be claimed regardless of the dependencies chosen in the attached claims.

### BRIEF DESCRIPTION OF THE DRAWINGS

In the drawings wherein like reference symbols/characters refer to like parts:

FIG. 1 shows two light sources L1 and L2 with different but overlapping spectrums 21 and 22, in accordance with various embodiments;

FIG. 2 shows one method of illuminating a sample sequentially (and optionally separately) at the same location with the two light sources L1 and L2, in accordance with various embodiments;

FIG. 3 is an example of an OCT system, and exemplary implemented as free space interferometers, in accordance with various embodiments;

FIGS. 4A to 4C illustrate characteristics of illuminating a sample sequentially at the same location with two or more light sources (e.g., L1 and L2), in accordance with various embodiments;

FIG. 5 is a diagram of an OCT system (OCT-2), in accordance with various embodiments;

FIG. 6 shows the spectral interference signals from the two interferometers (e.g., as produced by light sources L1 and L2), in accordance with various embodiments;

FIG. 7 shows the summation (e.g., combining) of the two modulated spectra, leading to a broader total modulated spectrum, in accordance with various embodiments;

FIG. 8 shows the magnitude of the Fourier transform of the L1 spectrum 51, the combined L1+L2 spectrum 53, and the smoothed combined spectrum 55 showing the resolution of the reflection in the sample arm, in accordance with various embodiments;

FIG. 9 shows signal summation after FFT prior to removing signal phase, in accordance with various embodiments;

FIG. 10 shows an example where light source L1 is comprised of a combination of multiple light sources (or sub light sources) L1a, L1b, and L1c, each having different spectra, in accordance with various embodiments;

FIGS. 11A to 11D show an example where light sources L1 and L2 are each comprised of multiple, individual light sources (or sub light sources), in accordance with various embodiments;

FIG. 12 illustrates optional methods for measurement of light returning from a sample using an interferometer, in accordance with various embodiments;

FIG. 13 provides an alternate embodiment incorporating a reference interferometer 77 into the reference arm R of an OCT system, in accordance with various embodiments;

FIG. 14 provides an example where each of multiple light sources, e.g., L1 and L2, has a respective reference interferometer 77 and 79, in accordance with various embodiments;

FIG. 15 shows the addition of a grating 81 in the source arm to deflect the angle of one light source (e.g., L1) relative to the other (e.g., L2) to overlap the light sources on the same scan, in accordance with various embodiments;

FIGS. 16A to 16C extend the graphs of FIGS. 4A to 4C to capture the effects of such a grating on the beams, showing the impact on the angle of the light illuminating the sample versus wavelength, in accordance with various embodiments;

FIGS. 17A-17C show the impact of bidirectional scanning on the use of grating to overlap light sources, in accordance with various embodiments;

FIG. 18 modifies the OCT system of FIG. 5 by the addition of waveplates and a Wollaston prism to provide balanced detection, in accordance with various embodiments;

FIG. 19 illustrates a generalized frequency domain optical coherence tomography system used to collect 3D image data of the eye suitable for use with the present disclosure, in accordance with various embodiments;

FIG. 20 shows an exemplary OCT B-scan image of a normal retina of a human eye, and illustratively identifies various canonical retinal layers and boundaries, in accordance with various embodiments;

FIG. 21 shows an example of an en face vasculature image, in accordance with various embodiments;

FIG. 22 shows an exemplary B-scan of a vasculature (OCTA) image, in accordance with various embodiments; and

FIG. 23 illustrates an example computer system (or computing device or computer), in accordance with various embodiments.

### DETAILED DESCRIPTION

The following detailed description of various embodiments herein makes reference to the accompanying drawings, which show various embodiments by way of illustration. While these various embodiments are described in sufficient detail to enable those skilled in the art to practice the disclosure, it should be understood that other embodiments may be realized and that changes may be made without departing from the scope of the disclosure. Thus, the detailed description herein is presented for purposes of illustration only and not for limitation. Furthermore, any reference to singular includes plural embodiments, and any reference to more than one component or step may include a singular embodiment or step. Also, any reference to attached, fixed, connected, or the like may include permanent, removable, temporary, partial, full, or any other possible attachment option. Additionally, any reference to without contact (or similar phrases) may also include reduced contact or minimal contact. It should also be understood that unless specifically stated otherwise, references to “a,” “an” or “the” may include one or more than one, and that reference to an item in the singular may also include the item in the plural. Further, all ranges may include upper and lower values, and all ranges and ratio limits disclosed herein may be combined.

Optical coherence tomography (OCT) is a non-invasive imaging technique that uses light waves to penetrate tissue and produce image information at different depths within the tissue, such as an eye. Generally, an OCT system is an interferometric imaging system based on detecting the interference of a reference beam and backscattered light from a sample illuminated by an OCT beam. Each scattering profile in the depth direction (e.g., z-axis or axial direction) may be reconstructed individually into an axial scan, or A-scan. Cross-sectional slice images (e.g., two-dimensional (2D) bifurcating scans, or B-scans) and volume images (e.g., 3D cube scans, or C-scans, or volume scans) may be built up from multiple A-scans acquired as the OCT beam is scanned/moved through a set of transverse (e.g., x-axis and/or y-axis) locations on the sample. When applied to the retina of an eye, OCT generally provides structural data that, for example, permits one to view, at least in part, distinctive tissue layers and vascular structures of the retina. OCT angiography (OCTA) expands the functionality of an OCT system to also identify (e.g., render in image format) the presence, or lack, of blood flow in retinal tissue. For example, OCTA may identify blood flow by identifying differences over time (e.g., contrast differences) in multiple OCT scans of the same retinal region, and designating as blood flow differences in the scans that meet predefined criteria.

An OCT system also permits construction of a planar (2D), frontal view (e.g., en face) image of a select portion of a tissue volume (e.g., a target tissue slab (sub-volume) or target tissue layer(s), such as the retina of an eye). Examples of other 2D representations (e.g., 2D maps) of ophthalmic data provided by an OCT system may include layer thickness maps and retinal curvature maps. For example, to generate layer thickness maps, an OCT system may combine en face images, 2D vasculature maps of the retina, with multilayer segmentation data. Thickness maps may be based, at least in part, on measured thickness difference between retinal layer boundaries. Vasculature maps and OCT en face images may be generated, for example, by projecting onto a 2D surface a sub-volume (e.g., tissue slab) defined between two selected layer-boundaries. The projection may use the sub-volume's mean, sum, percentile, or other data aggregation method between the selected two layer-boundaries. Thus, the creation of these 2D representations of a 3D volume (or sub-volume) data often relies on the effectiveness of automated (multi) retinal layer segmentation algorithm(s) to identify the retinal layers (or layer-boundaries) upon which the 2D representations are based/defined. Therefore, good axial resolution down to a desired (target) depth, such as 5 μm or more, is important to identify different tissue layers and achieve accurate images of the eye's structure and function.

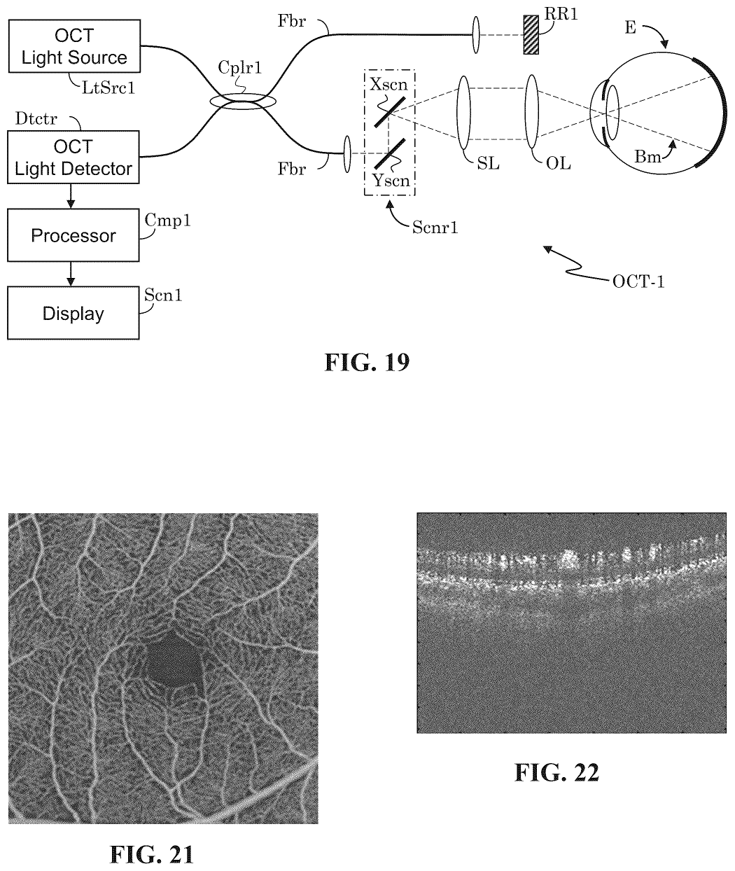

Various OCT architectures and their subcomponents are described below in detail, but for the sake of discussion, it is helpful to here provide a general description of a typical frequency domain optical coherence tomography (FD-OCT) system. With reference to FIG. 19, an FD-OCT system OCT_1 includes a light source, LtSre1. Typical light sources include, but are not limited to, broadband light sources with short temporal coherence lengths or swept laser sources. A beam of light from light source LtSrc1 is routed to illuminate a sample, e.g., select tissue within eye E. In the present example, light from light source LtSrc1 is routed by optical fiber Fbr1 to the sample to define a sample arm of an interferometer, and light from light source LtSrc1 is also routed by optical fiber Fbr2 to retroreflector RR1 to define a reference arm of the interferometer. However, the sample arm and reference arm in the interferometer could consist of bulk-optics (e.g., a free space interferometer) instead of fiber-optics, or could be a hybrid bulk-optic system, or incorporate micro optical bench technology or photonic integrated circuitry. The interferometer could also have different architectures such as Michelson, Mach-Zehnder or common-path based designs.

In the case of a spectral domain OCT (SD-OCT), the light source LrSrc1 may, for example, be a broadband light source with short temporal coherence length, and in the case of swept source OCT (SS-OCT), the light source LrSrc1 may be a wavelength tunable laser source. The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produces substantially collimated light, and ophthalmic lens OL may then focus the beam onto the sample. The direction of the beam toward the eye E is the axial direction (e.g., z direction) and a plane normal (perpendicular) to the axial direction defines x and y directions on a Cartesian plane. The present example illustrates a scan beam that is scanned in two lateral directions (e.g., in x and y directions) to scan a desired field of view (FOV). An example of this would be a point-field OCT, which uses a point-field beam to scan across a sample. Consequently, scanner Scnr1 is illustratively shown to include two sub-scanner: a first sub-scanner Xsen for scanning the point-field beam across the sample in a first direction (e.g., a horizontal x-direction); and a second sub-scanner Yscn for scanning the point-field beam on the sample in traversing second direction (e.g., a vertical y-direction).

Scattered light returning from the sample is collected into the same optical fiber Fbr1 used to route the light for illumination. Reference light derived from the same light source LtSrc1 travels a separate path, in this case involving optical fiber Fbr2 and retroreflector RR1 with an adjustable optical delay. The reference light returns from the retroreflector RR1. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, alternative designs of interferometers can be used for balanced or unbalanced detection of the interference signal, which may use more fiber ports to the detector. The output from the detector Dtctr1 is supplied to a processor (e.g., internal or external computing device) Cmp1 that converts the observed interference into depth information of the sample. The depth information may be stored in a memory and/or displayed on a display Scn1.

The example OCT system of FIG. 19 shows a single light source LtSrc1, and in the case of OCT_1 being a swept source OCT (SS-OCT), light source LtSrc1 would generally be a single, broad band, wavelength tunable laser. The present disclosure, however, introduces the use of multiple (e.g., two or more), narrower bandwidth lasers to achieve a similar axial (e.g., depth) resolution (e.g., of about 5 μm) as that achievable by a broad band, wavelength tunable laser. The cost of multiple narrower bandwidth lasers is less than that of a single broad band tunable laser, which leads to a more cost-effective OCT system design.

Of particular interest are light sources for use in a coherent interferometric measurement system, such as an OCT. In particular, herein is present a method and system for combining measurements obtained from multiple light sources to achieve a target axial resolution higher than that achievable by any single light source measurement, alone. The present disclosure is demonstrated as applied to a swept source OCT, but is to be understood that the present disclosure may be applied other types of OCT systems and other interferometric measurement systems.

As is well known in the art, the axial resolution of coherent imaging systems such as OCT is limited by the bandwidth of their illumination source. Unfortunately, the high cost of broad band-light sources adversely impacts the affordability of high-end OCT systems, particularly swept source OCT systems. A solution is to replace broad-band light source with multiple, inexpensive narrower-band light sources, enabling a more cost-effective swept source instrument. Here we show a means for sequentially illuminating a sample with two or more (cost-effective, narrow-band) light sources followed by mathematically combining the resulting signals to generate an image with a resolution supported by the combined spectrum of the two or more light sources. In effect, the combination of the two or more light sources achieves (or exceeds) the axial resolution of a single broad-band light source at a much reduced cost.

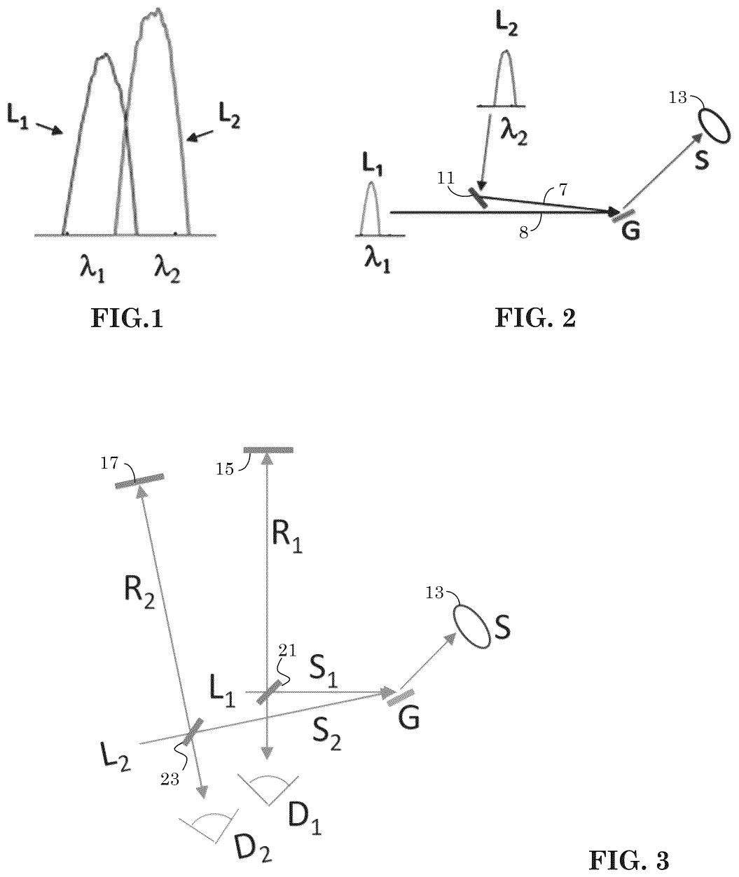

With reference to FIG. 1, two light sources L1 and L2 (e.g., two narrow-band light sources or of lower bandwidth than a traditional broad-band light source) with different but overlapping spectrums λ1 and λ2, respectively, are shown. It is desirable for the spectra of the illumination sources (e.g., L1 and L2) to be partially overlapping to enable a smooth spectrum when the signal is (mathematically) combined (e.g., summed, or combined by another data aggregation method or mathematical formulation). For example, the interferometrically measured signal (from both light sources L1 and L2) be summed coherently (e.g., the sum of amplitudes of spectrums, complex sum of spectrum or OCT amplitude if converted to complex data). Although only two (e.g., narrow-band) light sources L1 and L2 are shown, it is to be understood that more than two light sources may be used.

FIG. 2 shows one exemplary method of illuminating a sample sequentially at the same location with the two light sources L1 and L2. To achieve a resolution corresponding to the combined spectrum of the two light sources L1 and L2, one can illuminate the sample (not shown) sequentially (e.g., one light source at a time) at the same location with the two (or more) light sources through independent light paths 7 and 8, each of which defines respective sample beam (spatially offset from each other) along a sample arm S. The respective sample beams contact the sample at two (different) corresponding offset locations (e.g., scanned points), but as the respective sample beams are scanned (e.g., swept across the sample), each sample beam (such as from light path 8) follows the path of another (e.g., its neighboring) sample beam (such as light path 7) so that both scan beams traverse (and scan) the same scanned points/locations. In this manner, each scanned point/location may be scanned sequentially by light sources L1 and L2. Each sample beam thus provides a corresponding partial scan of the same scanned point/location, and both partial scans may then be combined to achieve the effective axial resolution of a single, broad-band light source. This can be achieved by bringing the light from the two light sources L1 and L2 onto a galvo mirror G (such as scanner Scnr1 of FIG. 19) with a slight angular displacement between them, and then rotating the galvo mirror. In the present example, light source L2 is directed onto light path 7 by a mirror 11 and conveyed to Galvo G, light source L1 is aligned directly to Galvo G, and the output from Galvo mirror G (L1 an L2) is directed along sample arm S to lens 13 (e.g., scan lens SL or ophthalmic or ocular lens OL of FIG. 19) to scan the sample. Although not shown in FIG. 2, it is to be understood that light from the light sources L1 and L2 is also directed to one or more collection arm, such as discussed in reference to FIG. 19 and shown in FIG. 3.

FIG. 3 provides an example of an OCT system similar to that of FIG. 19 in accord with the present disclosure, but diagramed as free space interferometers using bulk-optics instead of fiber-optics. In the present case, light from light sources L1 and L2 is divided by respective beam splitters 21 and 23 (serving a similar function as fiber coupler Cplr1 of FIG. 19) to define two respective reference arms R1 and R2 and two respective sample paths S1 and S2 that lead to sample arm S. Reference arms R1 and R2 lead to respective retroreflectors 15 and 17. For simplicity, both sample paths S1 and S2 lead to the same galvo G (although separate, respective galvos may alternatively be used), which directs their respective light to lens 13 along sample arm S to a sample to be scanned (and imaged). This diagram shows how light from the two sample paths S1 and S2 (and corresponding reference arms R1 and R2) can be collected interferometrically to generate the signals that are collected (e.g., by one or more detectors D1/D2), and then combined mathematically. First sample path S1 and first reference arm R1 may constitute a first interferometer, and second sample path S2 and second reference arm R2 may constitute a second interferometer. By contrast, a standard OCT system generally has the first but not the second interferometer. Optionally, each of first and second sample paths S1 and S2 may define separate sample arms if each has a separate path (e.g., separate galvo, scan lens, etc.) leading to the sample to be imaged. Signal collected from the two illuminations (e.g., by D1 and D2) is then combined mathematically to generate a high-resolution image.

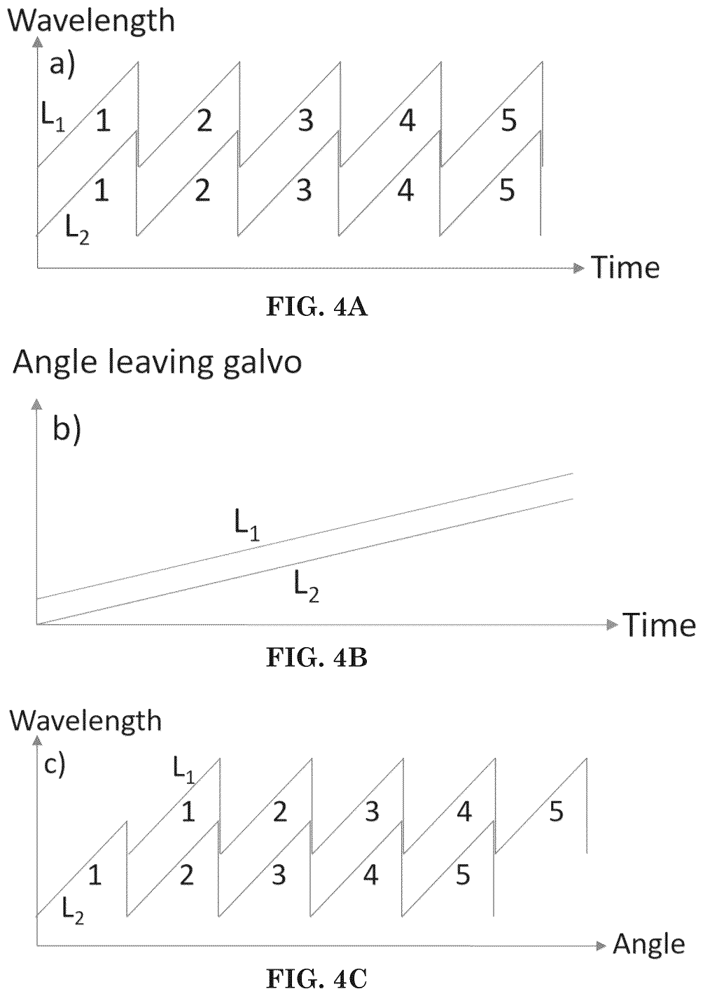

FIGS. 4A to 4C illustrate characteristics of illuminating a sample sequentially at the same location with light sources L1 and L2, as discussed above. Illuminating the sample in this way with repetitive swept sources will lead to the spectral illumination of the sample over time. FIG. 4A shows the wavelength of light sources L1 and L2 versus time, FIG. 4B shows the angle of light from light source L1 and L2 leaving galvo G versus time, and FIG. 4C plots the wavelength of light source L1 and L2 versus angle. In this example, the rotation rate of the galvo is set such that it shifts the angle of the beams during an A-scan by the displacement angle between the beams, thus causing the angle of beam 1 from the n+1 scan (e.g., of light source L1) to align with the angle of beam 2 from the nth scan (e.g., of light source L2) as shown in FIG. 4C. If the galvo were moving m times slower, then the alignment would be between the n+m^{th} and n^{th} scan, which would also work. Likewise, if the scan were moving in the opposite direction for bidirectional scanning, then the alignment would be between the n+m^{th} scan of beam 2 and the nth scan of beam 1. Thus, this design supports multiple options for the spacing between the scans in addition to bidirectional scanning.

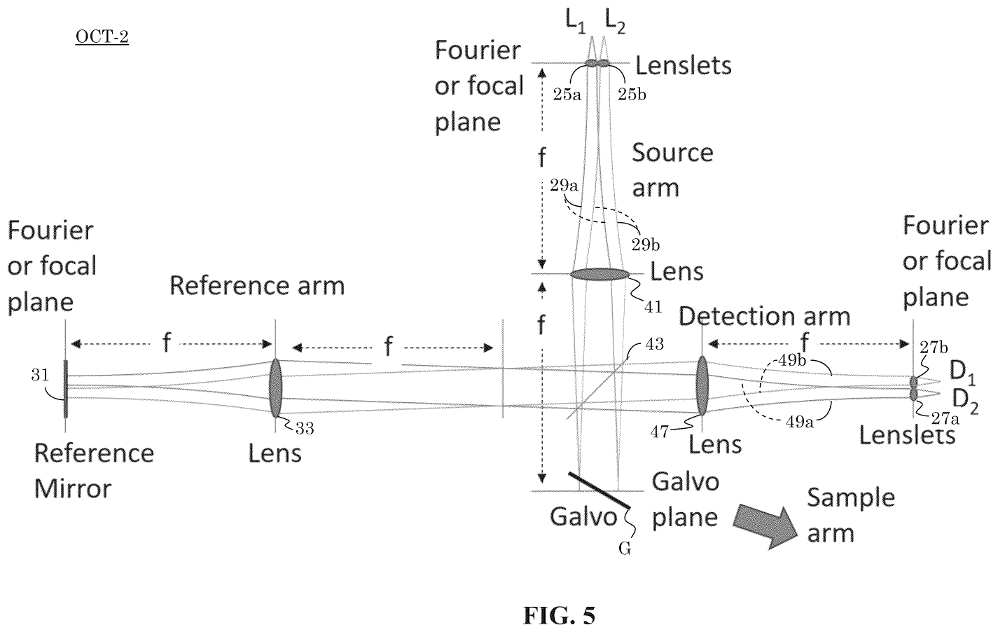

FIG. 5 is a diagram of an OCT system (OCT-2) in accord with the present disclosure. The present diagram shows two interferometers with shared optics and two light sources L1/L2 defining two light paths (e.g., 29a/29b and/or 49a/49b) separated by lenslet pairs (e.g., 25a/25b and 27a/07b) at the Fourier plan for the galvo G. Light from light sources L1/L2 is conveyed by first pair of lenslets 29a/29b and lens 41 to beam splitter 43, where the light beams from each of the light sources L1/L2 is split with part of each light beam going to a reference arm and the remainder going to galvo G and continuing to a sample arm to scan a sample. Reference mirror 31 (e.g., similar to retroreflectors 15 and 17 of FIG. 3) is also placed near the Fourier plane of the galvo(s) G, and optics (e.g., lens 33) are designed such that all the interferometer beams are perpendicular to the reference mirror 31. Light refracted from the sample and light returning from the reference arm are combined at beam splitter 43, where they interfere with each other and are conveyed to the detection arm. In the present case, a lens 47 focuses the respective light paths 49a/49b via a second pair of lenslets 27a/27b to respective detectors D1/D2. The second pair of lenslets 27a/27b may also substantially be at a Fourier or focal plane of the galvo (and/or first pair of lenslets 25a/25b). Note that additional lenslets could be added to increase the number of interferometric systems, either to support additional spectral bands, or to provide multiplexing to sample multiple locations on a sample (e.g., tissue) simultaneously.

OCT-2 is much like a classic OCT system in that it has a light source L1 and an interferometer is used for measuring the light returning from the sample. A difference is that OCT-2 uses a second OCT interferometer to collect the signal from a second light source L2. In general, the optimum place to separate the optical paths is where they are brought to a waist by focusing as this is the point where they have the greatest spatial separation. Although this is the optimum position, one could separate them at a slightly different plane, with this just including slightly larger angular separation between the beams. Lenslet pair 25a/25b is shown used for separating the beams, but other optics that affect one beam differently from the other could also be used, for instance an optical wedge across one beam, or a pair of wedges deflecting the two beams in different directions. The lenslets are desirable as they could transmit the light from adjacent light sources (as shown with lenslets 25a/25b) or two adjacent detectors (as shown with lenslets 27a/27b). Although these diagrams show only two interferometric paths, one could combine more than two light sources using the same, or similar, approach.

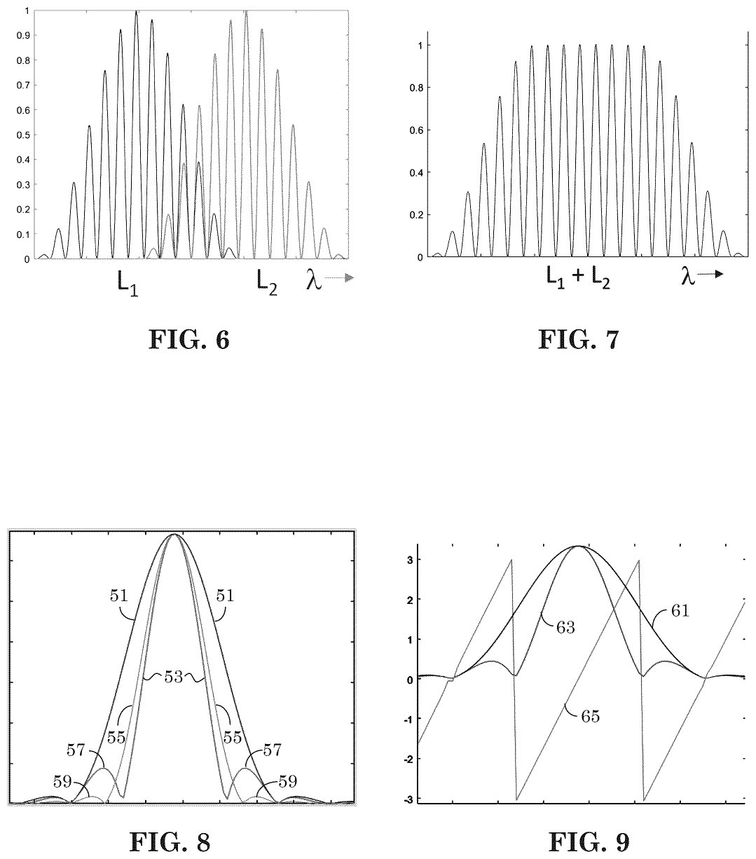

FIG. 6 shows the spectral interference signals from the two interferometers (e.g., as produced by light sources L1 and L2). Shown are interferometric signals from light sources L1 and L2 returning from the two interferometers for the case of a single reflector in the sample arm, with the interference leading to a modulation in the spectra.

FIG. 7 shows the summation (e.g., combining) of the two modulated spectra, leading to a broader total modulated spectrum. That is, FIG. 7 illustrates how when summed together, the spectral interference signals from the two interferometers provide an interference signal over a broader spectrum than either light source individually, thus enabling higher resolution. Taking the Fast Fourier Transform (FFT) of this spectrum and zooming in on the modulation frequency, as shown in FIG. 8, illustrates this narrowing of the resolution due to the broader bandwidth.

FIG. 8 shows the magnitude of the Fourier transform of the L1 spectrum 51, the combined L1+L2 spectrum 53, and the smoothed combined spectrum 55 showing the resolution of the reflection in the sample arm. Note that the combined spectrum 53 has a narrower Fourier transform corresponding to higher resolution, but has more pronounced side lobes 57. However, smoothing of the spectrum, as shown by the smoothed combined spectrum 55, reduces (or substantially eliminates) the side lobes 59 of this signal. Thus, one may also choose to smooth the envelope of the summed spectra to remove higher frequency components in the envelope that can lead to side lobes in the signal. Taking the FFT directly of L₁+L₂ leads to trace 53 with the sidelobes 57, while smoothing of the spectra produces curve 55 (substantially) without the sidelobes. As the FFT is a linear operation, one can also do this summing in the spatial domain after the FFT and prior to dropping the phase information, as shown in FIG. 9.

FIG. 9 shows summation after FFT prior to removing signal phase. Line 61 is the magnitude of FFT of both L1 and L2 spectrum, and line 65 is the phase difference between them. Resolution is narrowed by the two signals being roughly π radians out phase in the wings, resulting in the same (or similar) shape as when the summation is done in the spectral domain. Here, as both L₁ and L₂ had the same spectral shape, they have the same magnitude, shown as curve 61, but the second signal L₂ has a linear phase offset relative to L₁ as shown by curve 65, leading to the cancellation of signal in the wings that produces narrower curve 63 when the signals are summed.

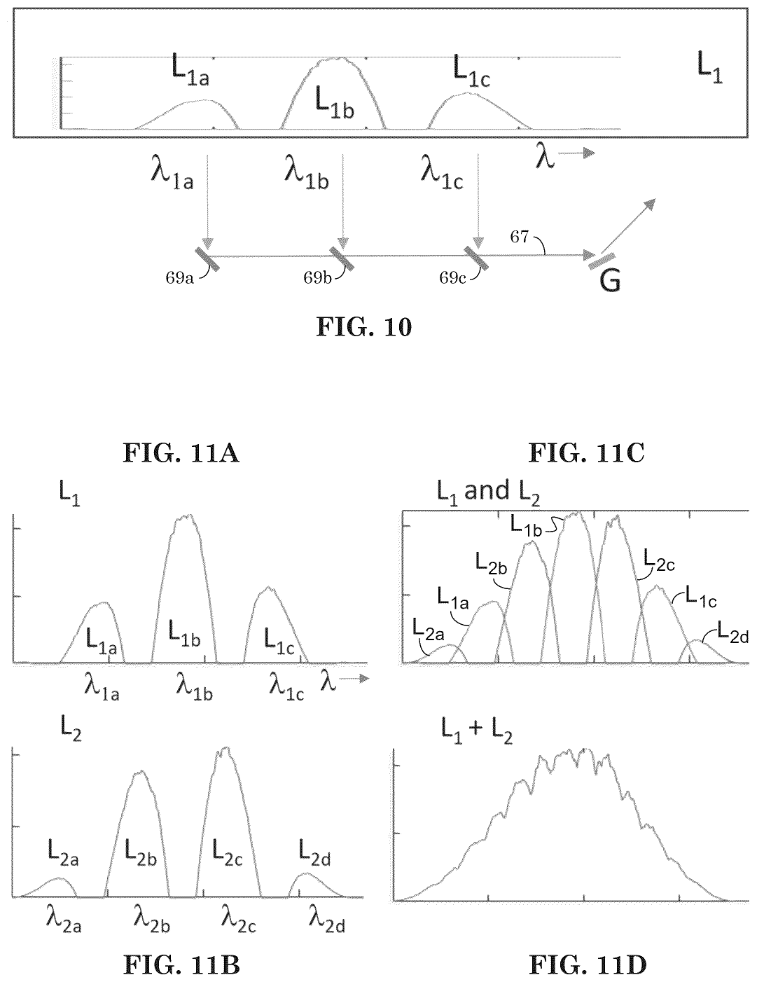

FIG. 10 shows an example where light source L1 is comprised of the combination of multiple light sources (or sub light sources) L1a, L1b, and L1c, each having different spectra. In the present example, light from light sources L1a, L1b, and L1c are combined by respective mirrors 69a, 69b, and 69c into a single optical path 67, which lead to Galvo G. To simplify both the combining of the light and the later separation of the signals, the individual light sources L1a, Lib, and L1c should ideally have non-overlapping spectral bands, as shown. Here, the light sources L1a, L1b, and L1c have separate spectra centered at wavelengths λ1a, λ1b, and λ1c, respectively.

FIGS. 11A to 11B show an example where light sources L1 and L2 are each comprised of multiple, individual light sources (or sub light sources). FIG. 11A shows L1 comprised of a first combination of multiple (e.g., three), individual light sources L1a, L1b, and L1c, and FIG. 11B shows light sources L2 similarly comprised of a second combination of multiple (e.g., four), individual light sources L2a, L2b, L2c, and dL2d. Similar to the light source example shown in FIG. 1, it is desirable to have two or more light sources with overlapping spectral regions as discussed above. FIG. 11C shows the spectrums of L1 and L2 superimposed on the same plot, and FIG. 11D shows the sum of the spectrums of L1 and L2. For optimum resolution, it is desirable to remove the ripple shown in FIG. 11D, which is caused by the summing of the spectra.

As shown in FIGS. 10 and 11, one could further extend the spectrum by combining multiple sources with different wavelength bands together into one or both illumination paths. In doing this, it is desirable that the light sources being combined into a given illumination have non-overlapping spectra, both to simplify the combining of the light sources (likely done through hot mirrors, or other optical components with spectral dependence), and particularly for the case where swept source illumination is used, to enable separation of the light from the individual sources during detection. One could operate the individual light sources in a given illumination path sequentially to separate the light between the individual sources. However, sequential illumination increases the total time to measure the tissue, thus reducing the A-scan rate. Operating the lasers simultaneously without separating the light prior to detection would both reduce the SNR (signal to noise ratio) and potentially create issues with separation of the signals. The loss in SNR is a result of having the reference light from multiple sources on the detector, thus increasing reference arm power and corresponding shot noise, without increasing signal.

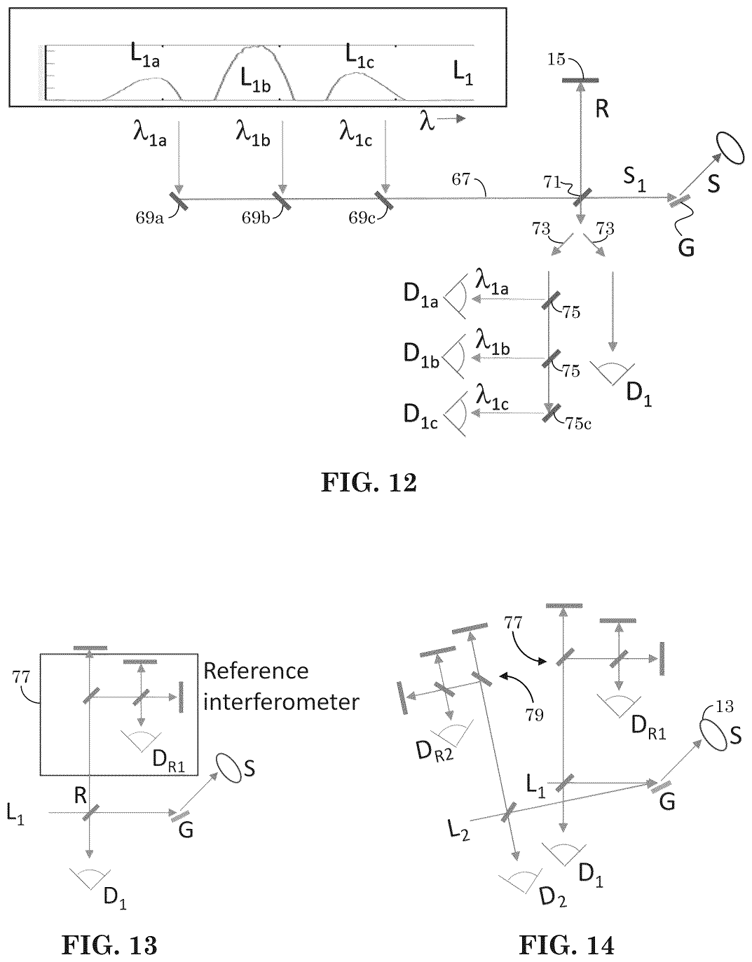

FIG. 12 illustrates optional methods for measurement of the light returning from the sample using an interferometer (hereby referenced as signal interferometer). As described above, light source L1 is again shown comprised of three sub light sources L1a, Lib, and L1c with non-overlapping spectra. Sub light sources L1a, L1b, and L1c are combined into optical path 67 by respective mirrors 69a, 69b, and 69c. Optical path 67 is here shown divided by beam splitter 71 into reference arm R leading to retroreflector (or mirror) 15, and into sample path S1 leading to Galvo G and sample arm S. Returning light from the reference arm R and sample arm S is then recombined to generate interferometric signals. In this diagram, the splitting and recombining are exemplarily done by the beamsplitter 71, but there are alternate interferometric configurations that can achieve this splitting and recombining function. FIG. 12 shows two interferometer configurations with different, optional detector paths 73a and 73b. In this manner, return light is either incident upon a single detector D1 along path 73b, or separated to be incident upon multiple, respective detectors D1a, D1b, D1c, as described above, along path 73a.

For example, one can operate all the sub light sources L1a, L1b, and L1c simultaneously and split the return light (e.g., the interferometric signal) into the different wavelength bands (e.g., using corresponding beam splitters 75a, 75b, 75c) to measure them separately by means of corresponding detectors D1a, D1b, and D1c, or one can operate sub light sources L1a, L1b, and L1c sequentially, enabling collection of the return light with a single detector D1. Operating them all simultaneously and detecting the light with a single detector is also possible, but has an impact on sensitivity. Alternatively, one could combine multiple sub light sources with overlapping spectra into a single illumination path, and then operate them sequentially, collecting the return light on a single detector. The primary disadvantages of such a solution would be the optical losses in combining the overlapping spectra and the reduction in acquisition rate driven by the desire to operate the light sources sequentially.

FIG. 13 provides an alternate embodiment incorporating a reference interferometer 77 into the reference arm R of an OCT system in according with the present disclosure. If the wavelength sweeping versus time of the light sources is non-linear or variable, it is desirable to add reference interferometer 77 for measuring the wavelength versus time of the sources. Similar to the case for the primary interferometer (e.g., as shown in FIG. 12), reference interferometer 77 could use either a single detector DR1 collecting all wavelengths, or multiple detectors corresponding to different wavelength bands.

FIG. 14 provides an example where each of multiple light sources, e.g., L1 and L2, has a respective reference interferometer 77 and 79. Here is presented an example of a complete configuration, with two light sources L1 and L2, each consisting of multiple combined sub lights sources (as discussed above), with corresponding signal and reference interferometers.

For swept source OCT systems, which is an exemplary application for the present disclosure, the wavelength of each light source is swept across its spectrum during an acquisition, and this sweeping is often non-linear and variable from acquisition to acquisition. This variability in wavelength versus time creates phase errors in the interferometric signal, which leads to loss of image resolution. To address this, a reference interferometer may be added to the system, as shown in FIG. 13. In a classic swept source OCT system, this reference interferometer only has a single wavelength passing through it at a given time, and the detector DR1 measures a sinusoidal oscillation caused by constructive and destructive interference as this wavelength changes. Any non-linearities in the sinusoidal oscillation indicate non-linearity in the sweep rate, which can then be corrected with information from the sinusoidal oscillation. In present case, there can be multiple wavelengths passing through the system simultaneously due to the multiple individual light sources in each illumination path, and there may be multiple sinusoidal oscillations superimposed. One can either measure all the oscillations on one detector, as shown above, or break the light into its spectral bands corresponding to the light sources, and measure each oscillation independently. In addition to measuring the non-linearity of each light source, the measurement of these oscillations can also provide information to phase match the signal interferometer interference signals from the various sources in a given illumination path and between illumination paths. FIG. 14 shows the schematic of the full system, with two illumination paths and two reference interferometers 77/79. Note that sharing of the reference interferometer optics is also possible, as discussed above.

With a single reference interferometer, some knowledge of the starting wavelength is helpful as the sinusoidal cycle repeats each time the wavenumber changes by 1/(2D_{L}) where D_{L} is the path length difference between the arms in the reference interferometer. To eliminate this uncertainty, one could also add a second reference interferometer in a given interferometer to provide an absolute measure of wavelength.

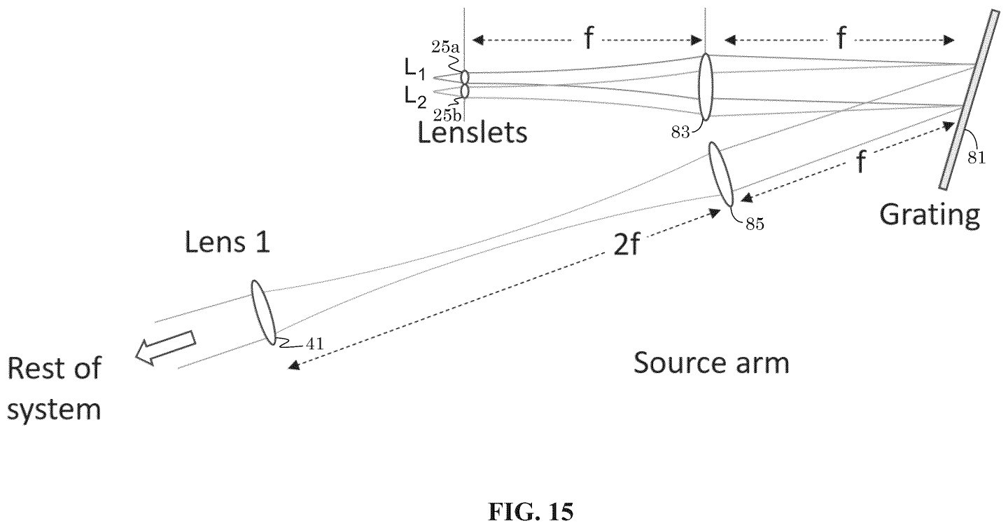

In an alternate embodiment, one could also use a grating or other dispersive element to combine the individual beams. FIG. 15, where elements similar to those of FIG. 5 have similar reference characters and are discussed above, shows the addition of a grating 81 (and lenses 83 and 85) in the source arm to deflect the angle of one light source (e.g., L1) relative to the other (e.g., L2) to overlap the light sources on the same scan. Here, the wavelength difference enables deflection of the angle of the second light source relative to the first, which is used to overlap the light sources on the same scan. In such a design, a dispersive element would also likely be used on the collection side both to separate the beams and to eliminate the variation in angle introduced in each beam by the dispersive element.

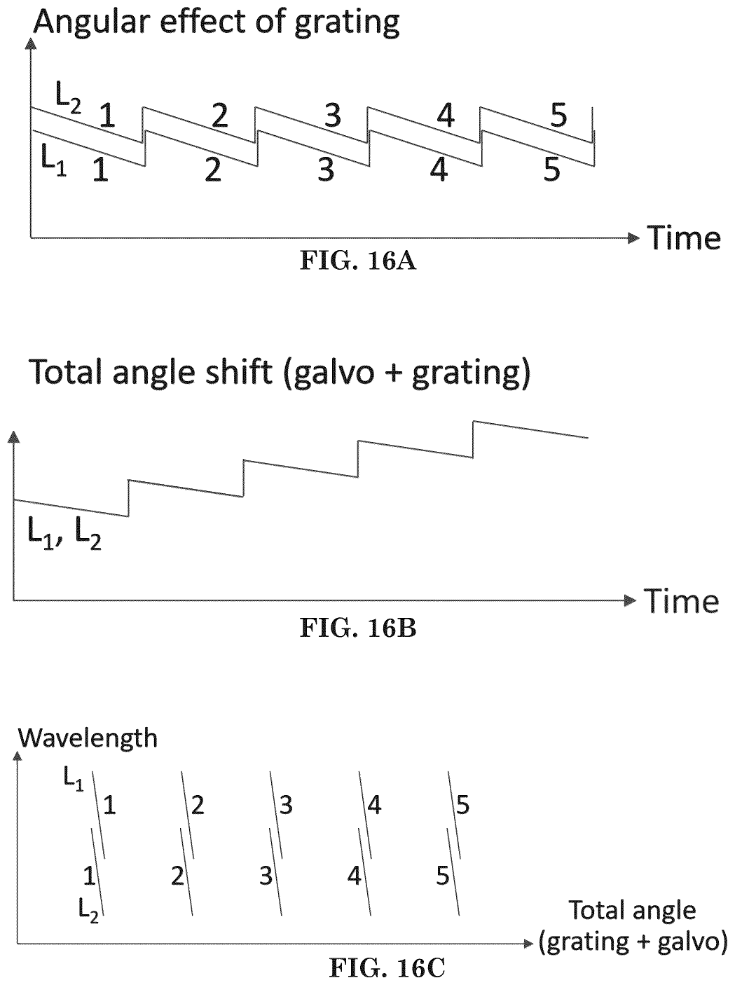

FIGS. 16A to 16C extend the graphs of FIGS. 4A to 4C to capture the effects of such a grating on the beams, showing the impact on the angle of the light illuminating the sample versus wavelength. FIG. 16A shows the angular effect of grating, where the sweeping of the wavelength by each light source, L1 and L2, leads to a variation in the angle leaving the grating. FIG. 16B shows the angle of light leaving the galvo, combining the effects of initial angular shift between beams, along with the effects of the rotating galvo mirror and grating. FIG. 16C shows the resulting angular shift versus wavelength for each light source, showing a result close to the ideal of all wavelengths for a given sweep having a fixed angular deflection independent of wavelength. The reason for the slight angular variation with wavelength is that the grating dispersion was chosen to cancel the angular displacement between the beams at the central wavelengths of the light sources, but the spectral extent of the light sources is slightly greater than the offset in central wavelengths to provide spectral overlap between the beams. One could choose the grating such that the wavelength sweep cancels the galvo rotation and then adjust the timing between the light source illuminations to make the illumination location independent of wavelength.

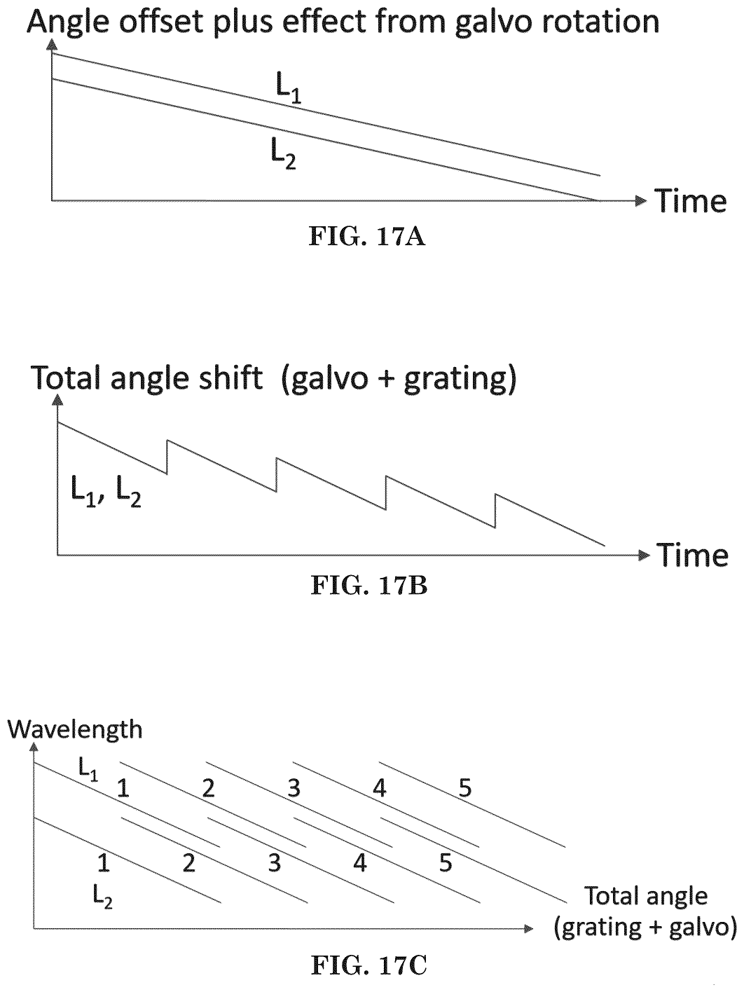

Although such a design can reduce the variation in angle of the spectrum for a given sweep, it can create complications for bidirectional scanning, because if one changes the sweep direction of the galvo or illumination source, the angular displacements no longer cancel. FIGS. 17A-17C show the impact of bidirectional scanning on the use of grating to overlap light sources. FIG. 17A is a plot of the angle offset versus time from scanning in opposite direction from FIGS. 4A-4C and 16A-16C. FIG. 17B shows the angular shift versus time for both light sources, and FIG. 17C shows angular shift versus wavelength for the two light sources. Note the large variation in angle for a given sweep. A similar effect can be seen if one flips the sweep direction of light sources rather than the galvo, and if both the light source and galvo sweep directions are reversed, the effects will cancel, leading back to a well-behaved system.

Although not directly addressed above, it is generally desirable to use balanced detection for swept source OCT systems. An example of balanced detection in an OCT interferometer design is provided in U.S. Pat. No. 7,126,693, assigned to the same assignee as the present disclosure. One may further use a Wollaston prism to separate the polarization states in the detection path. Generally, a Wollaston prism is an optical device that manipulates polarized light, e.g., it separates light into two separate linearly polarized outgoing beams with orthogonal polarization.

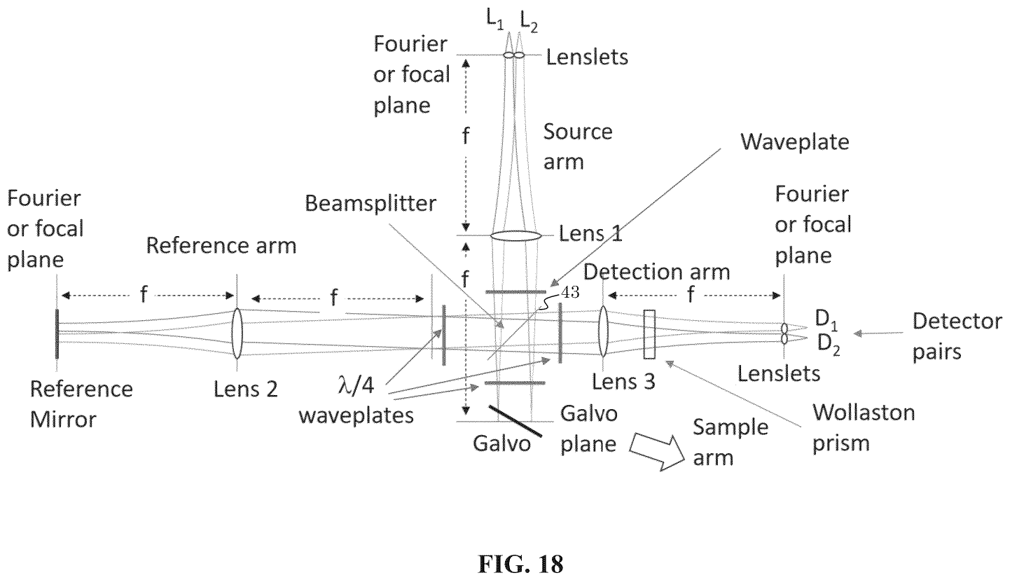

FIG. 18 modifies the OCT system of FIG. 5 by the addition of waveplates and a Wollaston prism to provide balanced detection. As it is known in the art, a waveplate, also known as a retarder, is an optical device that changes the polarization state of light passing through it without altering the beam's direction, displacement, or attenuation. Here the beamsplitter 43 is polarization dependent and the wave plates have the following operation. Waveplate in sample arm adjust the polarization of the source to select the fraction of light going to the reference. Waveplate in reference arm rotates the polarization state to maximize the light passing from the Reference arm to the Detection arm. Waveplate in Sample arm rotates polarization state to maximize light passing from Sample arm to Detection arm. Waveplate in detection arm adjusts polarization state such that the Wollaston prism splits the light equally into the two detection pairs.

Hereinafter is provided a description of various hardware and architectures suitable for the present disclosure.

### Optical Coherence Tomography Imaging System

Generally, optical coherence tomography (OCT) uses low-coherence light to produce two-dimensional (2D) and three-dimensional (3D) internal views of biological tissue. OCT enables in vivo imaging of retinal structures. OCT angiography (OCTA) produces flow information, such as vascular flow from within the retina. Examples of OCT systems are provided in U.S. Pat. Nos. 6,741,359 and 9,706,915, and examples of an OCTA systems may be found in U.S. Pat. Nos. 9,700,206 and 9,759,544, all of which are herein incorporated in their entirety by reference. An exemplary OCT/OCTA system is provided herein.

FIG. 19 illustrates a generalized frequency domain optical coherence tomography (FD-OCT) system used to collect 3D image data of the eye suitable for use with the present disclosure. An FD-OCT system OCT_1 includes a light source, LtSrc1. Typical light sources include, but are not limited to, broadband light sources with short temporal coherence lengths or swept laser sources. A beam of light from light source LtSrc1 is routed, typically by optical fiber Fbr1, to illuminate a sample, e.g., eye E; a typical sample being tissues in the human eye. The light source LrSrc1 may, for example, be a broadband light source with short temporal coherence length in the case of spectral domain OCT (SD-OCT) or a wavelength tunable laser source in the case of swept source OCT (SS-OCT). The light may be scanned, typically with a scanner Scnr1 between the output of the optical fiber Fbr1 and the sample E, so that the beam of light (dashed line Bm) is scanned laterally over the region of the sample to be imaged. The light beam from scanner Scnr1 may pass through a scan lens SL and an ophthalmic lens OL and be focused onto the sample E being imaged. The scan lens SL may receive the beam of light from the scanner Scnr1 at multiple incident angles and produces substantially collimated light, ophthalmic lens OL may then focus onto the sample. The present example illustrates a scan beam that is scanned in two lateral directions (e.g., in x and y directions on a Cartesian plane) to scan a desired field of view (FOV). An example of this would be a point-field OCT, which uses a point-field beam to scan across a sample. Consequently, scanner Scnr1 is illustratively shown to include two sub-scanner: a first sub-scanner Xsen for scanning the point-field beam across the sample in a first direction (e.g., a horizontal x-direction); and a second sub-scanner Yscn for scanning the point-field beam on the sample in traversing second direction (e.g., a vertical y-direction). If the scan beam were a line-field beam (e.g., a line-field OCT), which may sample an entire line-portion of the sample at a time, then only one scanner scans the line-field beam across the sample to span the desired FOV. If the scan beam were a full-field beam (e.g., a full-field OCT), no scanner may be needed, and the full-field light beam may be applied across the entire, desired FOV at once.

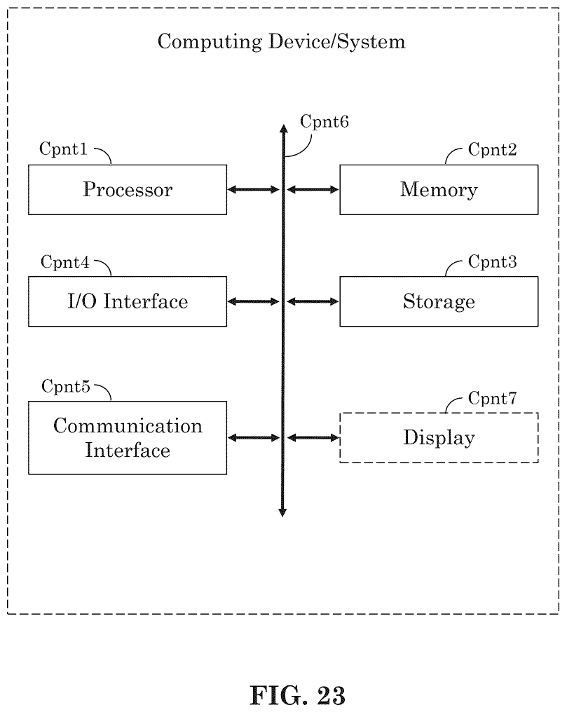

Irrespective of the type of beam used, light scattered from the sample (e.g., sample light) is collected. In the present example, scattered light returning from the sample is collected into the same optical fiber Fbr1 used to route the light for illumination. Reference light derived from the same light source LtSrc1 travels a separate path, in this case involving optical fiber Fbr2 and retroreflector RR1 with an adjustable optical delay. Those skilled in the art will recognize that a transmissive reference path can also be used and that the adjustable delay could be placed in the sample or reference arm of the interferometer. Collected sample light is combined with reference light, for example, in a fiber coupler Cplr1, to form light interference in an OCT light detector Dtctr1 (e.g., photodetector array, digital camera, etc.). Although a single fiber port is shown going to the detector Dtctr1, those skilled in the art will recognize that various designs of interferometers can be used for balanced or unbalanced detection of the interference signal. The output from the detector Dtctr1 is supplied to a processor (e.g., internal or external computing device) Cmp1 that converts the observed interference into depth information of the sample. The depth information may be stored in a memory associated with the processor Cmp1 and/or displayed on a display (e.g., computer/electronic display/screen) Scn1. The processing and storing functions may be localized within the OCT instrument, or functions may be offloaded onto (e.g., performed on) an external processor (e.g., an external computing device), to which the collected data may be transferred. An example of a computing device (or computer system) is shown in FIG. 23. This unit could be dedicated to data processing or perform other tasks which are quite general and not dedicated to the OCT device. The processor (computing device) Cmp1 may include, for example, a field-programmable gate array (FPGA), a digital signal processor (DSP), an application specific integrated circuit (ASIC), a graphics processing unit (GPU), a system on chip (SoC), a central processing unit (CPU), a general purpose graphics processing unit (GPGPU), or a combination thereof, that may performs some, or the entire, processing steps in a serial and/or parallelized fashion with one or more host processors and/or one or more external computing devices.

The sample and reference arms in the interferometer could consist of bulk-optics, fiber-optics, or hybrid bulk-optic systems and could have different architectures such as Michelson, Mach-Zehnder or common-path based designs as would be known by those skilled in the art. Light beam as used herein should be interpreted as any carefully directed light path. Instead of mechanically scanning the beam, a field of light can illuminate a one or two-dimensional area of the retina to generate the OCT data (see for example, U.S. Pat. No. 9,332,902; D. Hillmann et al, “Holoscopy—Holographic Optical Coherence Tomography,” Optics Letters, 36(13):2390 2011; Y. Nakamura, et al, “High-Speed Three Dimensional Human Retinal Imaging by Line Field Spectral Domain Optical Coherence Tomography,” Optics Express, 15(12):7103 2007; Blazkiewicz et al, “Signal-To-Noise Ratio Study of Full-Field Fourier-Domain Optical Coherence Tomography,” Applied Optics, 44(36):7722 (2005), which are hereby incorporated by reference in their entirety into this disclosure for all purposes). In time-domain systems, the reference arm may have a tunable optical delay to generate interference. Balanced detection systems are typically used in TD-OCT and SS-OCT systems, while spectrometers are used at the detection port for SD-OCT systems. The systems and methods described herein could be applied to any type of OCT system. Various aspects of the disclosure could apply to any type of OCT system or other types of ophthalmic diagnostic systems and/or multiple ophthalmic diagnostic systems including but not limited to fundus imaging systems, visual field test devices, and scanning laser polarimeters.

In Fourier Domain optical coherence tomography (FD-OCT), each measurement is the real-valued spectral interferogram (Sj(k)). The real-valued spectral data typically goes through several post-processing steps including background subtraction, dispersion correction, etc. The Fourier transform of the processed interferogram, results in a complex valued OCT signal output Aj(z)=|Aj|eiφ. The absolute value of this complex OCT signal, |Aj|, reveals the profile of scattering intensities at different path lengths, and therefore scattering as a function of depth (z-direction) in the sample. Similarly, the phase, φj can also be extracted from the complex valued OCT signal. The profile of scattering as a function of depth is called an axial scan (A-scan). A set of A-scans measured at neighboring locations in the sample produces a cross-sectional image (tomogram or B-scan) of the sample. A collection of B-scans collected at different transverse locations on the sample makes up a data volume or cube. For a particular volume of data, the term fast axis refers to the scan direction along a single B-scan whereas slow axis refers to the axis along which multiple B-scans are collected. The term “cluster scan” may refer to a single unit or block of data generated by repeated acquisitions at the same (or substantially the same) location (or region) for the purposes of analyzing motion contrast, which may be used to identify blood flow. A cluster scan can consist of multiple A-scans or B-scans collected with relatively short time separations at approximately the same location(s) on the sample. Since the scans in a cluster scan are of the same region, static structures remain relatively unchanged from scan to scan within the cluster scan, whereas motion contrast between the scans that meets predefined criteria may be identified as blood flow.

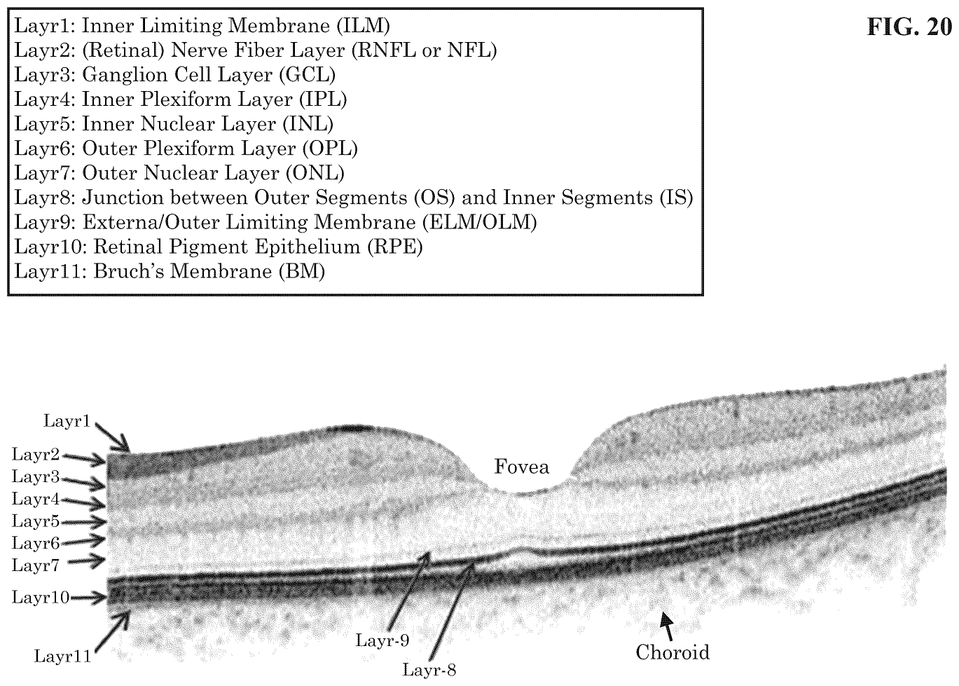

A variety of ways to create B-scans are known in the art including but not limited to: along the horizontal or x-direction, along the vertical or y-direction, along the diagonal of x and y, or in a circular or spiral pattern. B-scans may be in the x-z dimensions but may be any cross-sectional image that includes the z-dimension. An example OCT B-scan image of a normal retina of a human eye is illustrated in FIG. 20. An OCT B-scan of the retinal provides a view of the structure of retinal tissue. For illustration purposes, FIG. 20 identifies various canonical retinal layers and layer boundaries. The identified retinal boundary layers include (from top to bottom): the inner limiting membrane (ILM) Lyer1, the retinal nerve fiber layer (RNFL or NFL) Layr2, the ganglion cell layer (GCL) Layr3, the inner plexiform layer (IPL) Layr4, the inner nuclear layer (INL) Layr5, the outer plexiform layer (OPL) Layr6, the outer nuclear layer (ONL) Layr7, the junction between the outer segments (OS) and inner segments (IS) (indicated by reference character Layr8) of the photoreceptors, the external or outer limiting membrane (ELM or OLM) Layr9, the retinal pigment epithelium (RPE) Layr10, and the Bruch's membrane (BM) Layr11.

In OCT Angiography, or Functional OCT, analysis algorithms may be applied to OCT data collected at the same, or approximately the same, sample locations on a sample at different times (e.g., a cluster scan) to analyze motion or flow (see for example US Patent Publication Nos. 2005/0171438, 2012/0307014, 2010/0027857, 2012/0277579 and U.S. Pat. No. 6,549,801, which are hereby incorporated by reference in their entirety into this disclosure for all purposes). An OCT system may use any one of a number of OCT angiography processing algorithms (e.g., motion contrast algorithms) to identify blood flow. For example, motion contrast algorithms can be applied to the intensity information derived from the image data (intensity-based algorithm), the phase information from the image data (phase-based algorithm), or the complex image data (complex-based algorithm). An en face image is a 2D projection of 3D OCT data (e.g., by averaging the intensity of each individual A-scan, such that each A-scan defines a pixel in the 2D projection). Similarly, an en face vasculature image is an image displaying motion contrast signal in which the data dimension corresponding to depth (e.g., z-direction along an A-scan) is displayed as a single representative value (e.g., a pixel in a 2D projection image), typically by summing or integrating all or an isolated portion of the data (see for example U.S. Pat. No. 7,301,644, which is hereby incorporated by reference in its entirety into this disclosure for all purposes). OCT systems that provide an angiography imaging functionality may be termed OCT angiography (OCTA) systems.

FIG. 21 shows an example of an en face vasculature image. After processing the data to highlight motion contrast using any of the motion contrast techniques known in the art, a range of pixels corresponding to a given tissue depth from the surface of internal limiting membrane (ILM) in retina, may be summed to generate the en face (e.g., frontal view) image of the vasculature. FIG. 22 shows an exemplary B-scan of a vasculature (OCTA) image. As illustrated, structural information may not be well-defined since blood flow may traverse multiple retinal layers making them less defined than in a structural OCT B-scan, as shown in FIG. 20. Nonetheless, OCTA provides a non-invasive technique for imaging the microvasculature of the retina and the choroid, which may be critical to diagnosing and/or monitoring various pathologies. For example, OCTA may be used to identify diabetic retinopathy by identifying microaneurysms, neovascular complexes, and quantifying foveal avascular zone and nonperfused areas. Moreover, OCTA has been shown to be in good agreement with fluorescein angiography (FA), a more traditional, but more evasive, technique including the injection of a dye to observe vascular flow in the retina. Additionally, in dry age-related macular degeneration, OCTA has been used to monitor a general decrease in choriocapillaris flow. Similarly in wet age-related macular degeneration, OCTA may provide a qualitative and quantitative analysis of choroidal neovascular membranes. OCTA has also been used to study vascular occlusions, e.g., evaluation of nonperfused areas and the integrity of superficial and deep plexus.

### Computing Device/System

FIG. 23 illustrates an example computer system (or computing device or computer device). In some embodiments, one or more computer systems may provide the functionality described or illustrated herein and/or perform one or more steps of one or more methods described or illustrated herein. The computer system may take any suitable physical form. For example, the computer system may be an embedded computer system, a system-on-chip (SOC), a single-board computer system (SBC) (such as, for example, a computer-on-module (COM) or system-on-module (SOM)), a desktop computer system, a laptop or notebook computer system, a mesh of computer systems, a mobile telephone, a personal digital assistant (PDA), a server, a tablet computer system, an augmented/virtual reality device, or a combination of two or more of these. Where appropriate, the computer system may reside in a cloud, which may include one or more cloud components in one or more networks.

In some embodiments, the computer system may include a processor Cpnt1, memory Cpnt2, storage Cpnt3, an input/output (I/O) interface Cpnt4, a communication interface Cpnt5, and a bus Cpnt6. The computer system may optionally also include a display Cpnt7, such as a computer monitor or screen.

Processor Cpnt1 includes hardware for executing instructions, such as those making up a computer program. For example, processor Cpnt1 may be a central processing unit (CPU) or a general-purpose computing on graphics processing unit (GPGPU). Processor Cpnt1 may retrieve (or fetch) the instructions from an internal register, an internal cache, memory Cpnt2, or storage Cpnt3, decode and execute the instructions, and write one or more results to an internal register, an internal cache, memory Cpnt2, or storage Cpnt3. In particular embodiments, processor Cpnt1 may include one or more internal caches for data, instructions, or addresses. Processor Cpnt1 may include one or more instruction caches, one or more data caches, such as to hold data tables. Instructions in the instruction caches may be copies of instructions in memory Cpnt2 or storage Cpnt3, and the instruction caches may speed up retrieval of those instructions by processor Cpnt1. Processor Cpnt1 may include any suitable number of internal registers, and may include one or more arithmetic logic units (ALUs). Processor Cpnt1 may be a multi-core processor; or include one or more processors Cpnt1. Although this disclosure describes and illustrates a particular processor, this disclosure contemplates any suitable processor.

Memory Cpnt2 may include main memory for storing instructions for processor Cpnt1 to execute or to hold interim data during processing. For example, the computer system may load instructions or data (e.g., data tables) from storage Cpnt3 or from another source (such as another computer system) to memory Cpnt2. Processor Cpnt1 may load the instructions and data from memory Cpnt2 to one or more internal register or internal cache. To execute the instructions, processor Cpnt1 may retrieve and decode the instructions from the internal register or internal cache. During or after execution of the instructions, processor Cpnt1 may write one or more results (which may be intermediate or final results) to the internal register, internal cache, memory Cpnt2 or storage Cpnt3. Bus Cpnt6 may include one or more memory buses (which may each include an address bus and a data bus) and may couple processor Cpnt1 to memory Cpnt2 and/or storage Cpnt3. Optionally, one or more memory management unit (MMU) facilitate data transfers between processor Cpnt1 and memory Cpnt2. Memory Cpnt2 (which may be fast, volatile memory) may include random access memory (RAM), such as dynamic RAM (DRAM) or static RAM (SRAM). Storage Cpnt3 may include long-term or mass storage for data or instructions. Storage Cpnt3 may be internal or external to the computer system, and include one or more of a disk drive (e.g., hard-disk drive, HDD, or solid-state drive, SSD), flash memory, ROM, EPROM, optical disc, magneto-optical disc, magnetic tape, Universal Serial Bus (USB)-accessible drive, or other type of non-volatile memory.

I/O interface Cpnt4 may be software, hardware, or a combination of both, and include one or more interfaces (e.g., serial or parallel communication ports) for communication with I/O devices, which may enable communication with a person (e.g., user). For example, I/O devices may include a keyboard, keypad, microphone, monitor, mouse, printer, scanner, speaker, still camera, stylus, tablet, touch screen, trackball, video camera, another suitable I/O device, or a combination of two or more of these.

Communication interface Cpnt5 may provide network interfaces for communication with other systems or networks. Communication interface Cpnt5 may include a Bluetooth interface or other type of packet-based communication. For example, communication interface Cpnt5 may include a network interface controller (NIC) and/or a wireless NIC or a wireless adapter for communicating with a wireless network. Communication interface Cpnt5 may provide communication with a WI-FI network, an ad hoc network, a personal area network (PAN), a wireless PAN (e.g., a Bluetooth WPAN), a local area network (LAN), a wide area network (WAN), a metropolitan area network (MAN), a cellular telephone network (such as, for example, a Global System for Mobile Communications (GSM) network), the Internet, or a combination of two or more of these.

Bus Cpnt6 may provide a communication link between the above-mentioned components of the computing system. For example, bus Cpnt6 may include an Accelerated Graphics Port (AGP) or other graphics bus, an Enhanced Industry Standard Architecture (EISA) bus, a front-side bus (FSB), a HyperTransport (HT) interconnect, an Industry Standard Architecture (ISA) bus, an InfiniBand bus, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or other suitable bus or a combination of two or more of these.

Although this disclosure describes and illustrates a particular computer system having a particular number of particular components in a particular arrangement, this disclosure contemplates any suitable computer system having any suitable number of any suitable components in any suitable arrangement.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

While the disclosure includes several specific embodiments, it is evident to those skilled in the art that many further alternatives, modifications, and variations will be apparent in light of the foregoing description. Thus, this disclosure is intended to embrace all such alternatives, modifications, applications and variations as may fall within the spirit and scope of the appended claims.

1. A coherent interferometric measurement system, comprising:
two or more light sources each configured for emitting a different spectral wavelength band;
one or more beam divider configured for directing a first portion of each of the two or more light sources into one or more reference arm and a second portion of the two or more light sources into a sample arm;
optics configured for directing the light in the sample arm onto a sample;
one or more detector configured for receiving light returning from the sample and reference arms and generating signals in response thereto; and
a processor configured for converting the signals into image data;
wherein:
overlap exists between the spectral wavelength bands of the two or more light sources;
a location on a sample is illuminated with a first of the two or more light sources, followed by illumination with another of the two or more light sources; and
the light returning from the sample from each of the two or more light sources is measured with a coherent interferometric measurement system.

2. The system of claim 1, wherein the two or more measurements are summed coherently, including at least one of amplitudes of spectrums, complex sum of spectrum or optical coherence tomography (OCT) amplitude if converted to complex data.

3. The system of claim 1, wherein the two measurements are combined to provide a higher axial resolution than either measurement alone.

4. The system of claim 3, wherein the light sources sweep their wavelength across their respective spectral wavelength bands.

5. The system of claim 4, wherein the wavelength sweeping is non-linear in k, and the collected data is remapped to create spectra that are linear in k.

6. The system of claim 5, wherein the linearization in k is done prior to adding the signals together.

7. The system of claim 3, wherein the spectral overlap regions are used to measure a phase offset between the two signals.

8. The system of claim 7, wherein this phase offset is corrected prior to the summing of the two signals.

9. The system of claim 1, wherein at least one of the two or more light sources consists of at least two sub-sources each having a different respective spectrum.

10. The system of claim 9, wherein there is no overlap in the spectral bandwidth between the at least two sub-sources, and there is overlap between the spectral bandwidth of at least one of the at least two sub-sources and another of the two or more light sources.

11. The system of claim 1, wherein:
the optics configured for directing the light in the sample includes a scanner;
the second portions of the two or more light sources are brought onto the scanner with an angular displacement between them, each second portion defining a respective sample beam, spatially offset from each other, along the sample arm.

12. The system of claim 11, wherein the respective sample beams contact the sample at different corresponding offset locations; and
as the respective sample beams are scanned, each sample beam follows the path of another sample beam so that the sample beams traverse the same locations on the sample so that each offset location is scanned sequentially by a plurality of the sample beams.

13. The system of claim 1, wherein at least one of the two or more light sources includes one or more VCSELs, and the wavelengths of the VCSELs are swept by thermal heating.

14. The system of claim 1, wherein at least one of the two or more light sources includes one or more VCSELs, and the wavelengths of the VCSELs are swept by movement of a membrane mirror.

15. The system of claim 1, wherein a light source consists of one or more distributed-feedback laser (DFB) or Distributed Bragg reflector (DBR) tunable lasers.

16. The system of claim 1, wherein each of the one or more detectors are configured to receive respective light returning from the sample and reference arms corresponding to a respective one of the two or more light sources.

17. A coherent interferometric measurement system, comprising:
a first light source configured for generating a first beam of light having a first spectral wavelength band;
a second light source configured for generating a second beam of light having a second spectral wavelength band different than the first spectral wavelength band, the second spectral wavelength band overlapping the first spectral wavelength band;
optics configured for directing at least a portion of the first beam to illuminate a target location on a sample;
the optics configured for directing at least a portion of the second beam to illuminate the target location on the sample following illumination of the target location by the first beam in sequence;
one or more detectors configured for receiving light returning from the target location on the sample due to the first beam and generating first signals in response thereto;
the one or more detectors configured for receiving light returning from the target location on the sample due to the second beam and generating second signals in response thereto;
a processor configured for mathematically combining the first signals and second signals into a combined interferometric measurement for the target location on the sample; and
the processor configured for converting the combined interferometric measurement into image data.

18. The system of claim 17, wherein the coherent interferometric measurement system is an optical coherence tomography (OCT) system, further comprising:
one or more beam dividers configured for directing a first portion of the first beam into a first reference arm and a second portion of the first beam into a first sample arm;
the one or more beam dividers configured for directing a first portion of the second beam into a second reference arm and a second portion of the first beam into a second sample arm;
the optics direct second portion of the first beam in the first sample arm illuminates the target location;
the optics direct second portion of the second beam in the second sample arm illuminates the target location;
the one or more detectors configured to generate the first signals in response to returning light in the first sample arm and first reference arm; and
the one or more detectors configured to generate second signals in response to returning light in the second sample arm and second reference arm.

19. The system of claim 18, wherein the first reference arm is different than the second reference arm.

20. The system of claim 18, wherein the first sample arm and the second sample arm share optical components and light path to the target location on the sample.
